(19) United States
(12) Patent Application Publication
(10) Pub. No.: US 2009/0012163 A1
(43) Pub. Date: Jan. 8, 2009

(54) Compositions containing Mg/Zn/F-CaP plus inhibitors of pro-inflammatory cytokines (alpha ketoalkanoic acid compound) for osteoporosis prevention, therapy and treatment of bone diseases

(76) Inventor: Maria C. Gaffar, Princeton, NJ (US)

Correspondence Address: Allen R. Kipnes;WATOV & KIPNES, P.C., P. O. Box 247, Princeton Junction, NJ, 08550, US

(21) Appl. No.: 11/824,346

(22) Filed: Jul. 2, 2007

(51) Int. Cl. A61K 31/22; A61P 19/08

(52) U.S. Cl. 514/557

(57) ABSTRACT

A method is described for the treatment of bone disease by oral dosing of a patient with an inhibitor(s) or pro-inflammatory Cytokines which are comprised of a combination of alpha ketoalkanoic acid admixed in a pharmaceutically acceptable carrier with calcium/phosphate/magnesium/zinc or strontium salt compound ion—a hydroxyapatite matrix.

### FIELD OF THE INVENTION

This invention releates to the method for the treatment of bone disease wherein an alpha ketoalkanoic acid compound is admixed in a pharmaceutically acceptable carrier with calcium/phosphate/magnesium/zinc or strontium salt compound ion in a hydroxyapatite matrix. In the treatment of the patient.

### THE PRIOR ART

Prior art describes the use of fluoride (in the form of sodium fluoride, NaF or monofluorophosphate, MFP) for osteoporosis therapy. Studies showed that fluoride (F) stimulates bone forming cell in vitro but animal and clinical studies have shown that the effect of F is biphasic: at low concentration it increases bone mass and at high concentration causes bone fractures [Kleerecoper, 1999]. U.S. Pat. Nos. 3,306,824 and 4,265,877 disclosed F in the range of 0.25 to 1.0 mg per day. However these preparations cause gastric irritations. A sustained release preparation with calcium has been proposed to reduce irritations (U.S. Pat. Nos. 3,287,219; 4,130,630; 3,345,265; and 4,861,590.

There is a need for non-irritating low dose highly effective preparation for the treatment and prevention of osteoporosis. It has been demonstrated that magnesium (Mg) increase bone mass in humans [Dimai et al 1998] and zinc stimulated bone formation [Yamaguchi 1995]. WO05032466A (Apr. 14, 2005) by LeGeros describes combined concentrations of Mg, Zn and F ions in a carbonate containing calcium phosphate (CaP) system for osteoporosis prevention, therapy and for bone repair. The Mg/Zn/F—CaP preparations have the following advantages: (1) no serious side effects or deleterious effects on bone strength and structure unlike currently FDA approved anti-resorption agents; (2) compositions are similar to bone mineral which is a carbonate apatite containing small amounts of Mg and trace amounts of Zn (Mg and Zn ions are essential components of more than 200 enzyme systems in human); (3) allow slow release of Mg, Zn, F, Ca and P ions; (4) combine ions in concentrations that promote bone formation and minimize bone resorption; (5) allow the incorporation of lower levels of these ions to avoid deleterious side effects found with higher levels; (6) Mg and Zn ions have beneficial effects on collagen and protein formation and balance the F effect on bone apatite formation and crystal size to promote bone formation with higher mineral density and greater bone mass; (7) the three ions have a synergistic effect that allow the rate of bone formation to catch up with the rate of bone resorption, resulting in a net gain of bone mass.

Preliminary results with Mg/Zn/F—CaP in two FDA accepted models for osteoporosis (i.e., mineral-deficiency induced osteoporosis and ovariectomy induced osteoporosis) showed improvement in bone density, bone strength, cortical bone and trabecular bone thickness [LeGeros et al, AADR 2006; LeGeros reports to NIBIB/NIH, 2005].

The recent developments in the understanding of osteoporosis indicate that estrogen deficiency in post-menopausal women is the major cause of osteoporosis and estrogen has anti-inflammatory effects and inhibits the production of pro-inflammatory bone resorptive cytokines such as IL-1, TNF-_?, IL-6 [Jika et al 1992]. The functional block of TNF, but not IL-6, prevented bone loss in ovariectomized mice [J Bone Min Res 12:935-941, 1997]. On the other hand, estrogen deficiency results in the elevation of bone marrow production of pro-osteoclastogenic cytokines such as TNF. Currently available TNF and cytokine activation inhibitors uses monoclonal antibodies to block and require parenteral injections with sometimes serious side effects and are not practical for everyday use.

### SUMMARY OF THE INVENTION

### [Method]

A safe additive agent to Mg/Zn/F—CaP preparations is comprised of an alpha-ketoalkanoic acid, a C₃-C₈ straight chain or branched chain alpha-ketoalkanoic acid ester as described in patent applications WO02/07/740,301 and PTC/US02/08283 is effective to reduce the release of TNFa, a cytokine involved in inducing bone loss, which applications are herein incorporated by reference.

### DESCRIPTION of the PREFERRED EMBODIMENTS

The alpha-ketoalkanoic acid ester preferably, the therapeutic agent used in the method disclosed herein is an effective amount of an ester of an alpha-ketoalkanoic acid, for example, a C₃-C₈ straight-chained or branched alpha-ketoalkanoic acid ester. Examples include alpha-keto-butyrate, alpha-ketopentanoate, alpha-keto-3-methyl-butyrate, alpha-keto-4-methyl-pentanoate or alpha-keto-hexanoate. Pyruvate is preferred. A variety of groups are suitable for the ester position of the molecule, e.g., alkyl, aralkyl, alkoxyl, carboxyalkyl, glyceryl or dihydroxy acetone. Specific examples include ethyl, propyl, butyl, carboxymethyl, acetoxymethyl, carbethoxymethyl and ethoxymethyl. Ethyl esters are preferred. Thioesters (e.g. wherein the thiol portion is cysteine or homocysteine) are also included.

In a preferred embodiment, the pharmaceutical composition used in the disclosed method comprises ethyl pyruvate, propyl pyruvate, carboxymethyl pyruvate, acetoxymethyl pyruvate, carbethoxymethyl pyruvate, ethoxymethyl pyruvate, ethyl alpha-keto-butyrate, ethyl alpha-keto-pentanoate, ethyl alpha-keto-3-methyl-butyrate, ethyl alpha-keto-4-methyl-pentanoate, or ethyl alpha-keto-hexanoate. Ethyl pyruvate is most preferred. In yet another aspect, the therapeutic agent used in the method disclosed herein is an effective amount of an amide of an alpha-ketoalkanoic acid. Suitable amides of alpha-ketoalkanoic acids for use in the method of the present invention includes compounds having the following structural formula: RCOCONR¹R². R is an alkyl group; R¹ and R² are independently BH, alkyl, aralkyl, alkoxyalkyl, carboxyalkyl or BCHR³COOH; and R³ is the side chain of a naturally occurring amino acid. Preferably, the amide of an alpha-ketoalkanoic acid is a pyruvamide.

In another aspect, when the ester derivative is used, a component for inducing and stabilizing the enol resonance form of the ester at physiological pH values is included in the composition. The stabilizing component is a cationic material, preferably a divalent cation, and most preferably strontium, calcium or magnesium. This composition further comprises a pharmaceutically acceptable inert carrier substance in which the enol resonance form of the ester is stabilized at physiological pH values. The preferred salts are strontium citrate, gluconate, salicylate, carbonate, malate, tartrate, chloride, phosphate, sulfate, sulfonate, benzoate, carboxylate, gallate, lactate, laurate, valerate and selenide.

### EXAMPLES OF THE INVENTION

### Example I

The Effect of Alpha-Ketoalkanoic Acid on TNFa Release from Stimulated Peripheral Blood Monocytes (PBMCS)

The cells were cultured in RPMI 1640 supplemented with 1% serum albumin for approximately 12 hours before treating with LPS (Lipopolysaccharide from E. Coli) at increasing concentrations to induce TNFa release. A wide range from 3 to 100 micrograms per ml indicated that this mixture is highly effective in releasing a key cytokine involved in bone loss. Concentration of 10 micrograms/per ml showed the highest release of TNF. The effects of ethyl pyruvate (EP) on PBMC=s macrophages cultures with 5-10 micrograms of LPS was used to stimulate relase of TNFa. The table below shows that EP at different concentrations inhibited the release of TNFa when LPS & EP were incubated in the cultures. Thus EP is an effective inhibitor of TNFa release.

Inhibition by Ethyl Pyruvate of TNFa Release (Measured via ELISA Assay)

| | LPS | % | | % |
|---|---|---|---|---|
| Conc. (mM) EP | (5 ug/ml)* | Inhibition | LPS (10 ug/ml)* | Inhibition |
| 0 | 510 | 0 | 750 | 0 |
| 5 | 450 | 12 | 480 | 6 |
| 10 | 275 | 47 | 310 | 59 |
| 25 | 201 | 51 | 190 | 75 |
| 50 | 200 | 51 | 180 | 76 |

*arbitrary fluorescence units via ELISA

### Example II

Preparations Containing Mg/Zn/F—CaP with Alpha-ketoalkanoic Acid

Examples of regular tablets and sustained release tablets are described below. No sintering or heating is required for regular tablets. The Mg/Zn/F—CaP plus flavans alpha-ketoalkanoic acid are from 0.0001 to 10.0%)

The means for providing controlled (i.e., sustained) release of the active ingredient may be selected from any of the known sustained-release oral drug delivery system. Of the known sustained-release delivery systems, the preferred system is the Forest Synchtron drug delivery system in which the active ingredient is dispersed uniformly and homogenously throughout a matrix of water swellable modified cellulosic powder or fibers forming coherent network, as a matrix.

Soft gelatin capsules are prepared as follows:

| Mg/Zn/F—CaP plus alpha-ketoalkanoic acid* | 0.0001 to 10% | |
|---|---|---|
| Strontium citrate, gluconate, malate, or tartrate | 0.0015 | mg to 5% |
| Propyl gallate | 0.020 | mg |
| Vitamin E (dl-alpha tocopherol) | 0.020 | mg |
| Alcohol, dehydrated | 1.145 | mg |
| Vegetable oil | 98.800 | mg |
| Total fill weight per capsule: | 100.0 | mg |

*Includes 10% excess

### Compressed Tablet

Mg/Zn/F—CaP/alpha-ketoalkanoic acid combinations are adsorbed onto a suitable substrate (cellulosic material) and progressively diluted (triturated) and dispersed in a calcium carbonate (CaCO3) granules containing the equivalent of 250 mg to 500 mg of calcium. The granules are compressed into a tablet.

### Film-Coated Tablet

Mg/Zn/F—CaP/alpha-ketoalkanoic acid combinations are dispersed or dissolved in an organic solvent or aqueous mix, admixed with a pharmaceutically acceptable polymer and spray coated onto a table core containing CaCO3 or strontium carbonate (in an amount equivalent to 250 mg to 500 mg of calcium or strontium). Additional protective coating may also be added.

### Dry-Filled Capsule

Mg/Zn/F—CaP/alpha ketoalkanoic acid combinations are adsorbed onto a suitable substrate and placed in a capsule. The remainder of the capsule is filled with a suitable form of CaCO3 or strontium carbonate (containing the equivalent of 250 mg to 500 mg of calcium or strontium).
Microencapsulated amounts of 5-40 ug (mcg) of Mg/Zn/F—CaP/alpha ketoalkanoic acid combinations are placed in a capsule and the remainder of the capsule is filled with a suitable form of CaCO3 or strontium carbonate (containing the equivalent of 250 mg to 500 mg of calcium or strontium).
A pellet or tablet containing 5-40 ug (mcg) of Mg/Zn/F—CaP/alpha-ketoalkanoic acid combinations are placed in a capsule and the remainder of the capsule is filled with a suitable form of CaCO3 or strontium carbonate (containing the equivalent of 250 mg to 500 mg of calcium or strontium).

### Soft Gelatin Capsules

A non-aqueous slurry containing 5-40 ug (mcg) of Mg/Zn/F—CaP/alpha-ketoalkanoic acid combination is filled into a soft gelatin capsule.

### Liquid-Filled Hard Capsules

A non-aqueous slurry containing 5-40 ug (mcg) of Mg/Zn/F—CaP/alpha-ketoalkanoic acid combination is filled into a hard gelatin capsule.

### Granules/Powders

A suitably flavored granulate containing 5-40 ug (mcg) of Mg/Zn/F—CaP/alpha-ketoalkanoic acid combination is filled into a sachet. This formulation is suitable for dispersing in a glass of beverage (e.g., water, milk or fruit juice) for immediate consumption. A unit dose cup containing 5-40 ug (mcg) of Mg/Zn/F—CaP/alpha-ketoalkanoic acid combination and CaCO3 or strontium carbonate (containing the equivalent of 250 mg to 500 mg of calcium or strontium) in granulate/powder form comprising also flavors, thickeners and possibly other excipients. The product is suitable for reconstitution with water to be shaken and drunk as a suspension.

What is claimed is:

1. A method for the treatment of bone disease which comprises dosing the patient with an alpha ketanoic acid compound, selected from an alpha ketoalkanoic acid, a physiologically acceptable salt of alpha ketoalkanoic acid, an ester of an alpha ketoalkanoic acid or an amide of an alpha ketoalkanoic acid in a pharmaceutically acceptable carrier with calcium/phosphate/magnesium/zinc or strontium salts in a hydroxyapatite matrix.

2. The method of claim 1, wherein said alpha ketoalkanoic acid compound is C3 to C8, straight chained or branched. The composition of claim 1 wherein said alpha ketoalkanoic acid compound may be selected from the group consisting of alpha-keto-butyrate, alpha-ketopentanoate, alpha-keto-3-methyl-butyrate, alpha-keto-4-methyl-pentanoate or alpha-keto-hexanoate.

3. The method of claim 1, wherein said alpha-ketoalkanoic acid is pyruvic acid.

4. The method of claim 1 wherein said physiologically-acceptable salt may be selected from the group consisting of Na⁺, K⁺, Ca⁺⁺, Mg⁺⁺, Zn++ and Sr₊₊₊.

5. The method of claim 1, wherein said alpha-ketoalkanoic acid compound may be selected from the group consisting of alkyl, aralkyl, alkoxyl, carboxyalkyl, glyceryl or dihydroxy acetone.

6. The method of claim 1, wherein, said ester of alpha-ketoalkanoic acid compound may be selected from the group of consisting of ethyl, propyl, butyl, carboxymethyl, acetoxymethyl, carbethoxymethyl and ethoxymethyl.

7. The method of claim 1 wherein said ester of alpha-ketoalkanoic acicompound is an ethyl pyruvate.

8. The method of claim 1, wherein said amide of alpha-ketoalkanoic acid RCOCONR¹R², compound has the structural formula, wherein said formula comprises RCOCONR¹R²
Wherein
i. R is an alkyl group;
ii. R¹ and R² are independently BH, alkyl, aralkyl, alkoxyalkyl, carboxyalkyl or —CH R³COOH; and
iii R³ is the side chain of a naturally occurring amino acid.

9. The method of claim 1 wherein said amide of an alpha-ketoalkanoic acid compound is a pyruvamide.

10. The method of claim 1 wherein said carrier substance further comprises a biologically safe component for inducing and stabilizing enolization of the alpha-keto functionality of said acid at physiological pH values.

11. The method of claim 10, wherein said component for inducing and stabilizing enolization of the alpha-keto functionality of said ester is an inorganic, divalent cation.

12. The method of claim 11, wherein said divalent cation is calcium, strontium or magnesium.

13. The method of claim 1, wherein said alpha-ketoalkanoic acid portion of said ester is alpha-ketopropionic acid.

14. The method of claim 1, wherein said alpha-ketoalkanoic acid ester compound is ethyl pyruvate, said divalent cation is calcium or strontium.

15. The method of claim 1, wherein said alpha-ketoalkanoic acid ester compound is selected from the group consisting of ethyl 2-ketobutyrate, ethyl 2-ketopentanoate, ethyl 2-keto-3-methyl-butyrate, ethyl 2-keto-4-methyl-pentanoate and ethyl 2-ketohexanoate.

16. The method of claim 1, wherein said alpha-ketoalkanoic acid ester compound is admixed in a tablet, said tablet containing a cation selected from the group consisting of calcium, magnesium and strontium.
